(19) United States
(12) Patent Application Publication
(10) Pub. No.: US 2007/0122840 A1
(43) Pub. Date: May 31, 2007

(54) Assay panel comprising food allergens

(76) Inventor: Peter David George Cousins, Osbaldwick (GB)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 10/555,967

(22) PCT Filed: May 5, 2004
(86) PCT No.: PCT/GB04/01945

§ 371 Date: Dec. 4, 2006

(30) Foreign Application Priority Data
May 8, 2003 (GB) 0310522.8

(51) Int. Cl. G01N 33/53; C12M 3/00

(52) U.S. Cl. 435/7.1; 435/287.2

(57) ABSTRACT

There is described a substrate to which is applied, coupled or cross-linked to at least one part of said substrate an allergen wherein said allergen is a protein derived from an extract selected from at least one of the following food groups: cereal, legume, cocoa bean, nut, fruit, vegetable, shellfish, fish, yeast, dairy product, egg and meat.

### CROSS-REFERENCES TO RELATED APPLICATIONS

The present application is a U.S. national phase application of PCT Application No. PCT/GB2004/001945, filed May 5, 2004, and claims the benefit of Great Britain Application No. 0310522.8, filed May 8, 2003, each of which is incorporated herein by reference in its entirety for all purposes.

### FIELD OF THE INVENTION

The invention relates to an immunoassay for the detection of antibodies which bind food allergens the presence of which is linked to food intolerance. The invention also relates to a substrate for use with said immunoassay.

### BACKGROUND OF THE INVENTION

A number of chronic pathological conditions are thought to be caused or provoked by allergic reactions to allergens present in food. For example, conditions such as irritable bowel syndrome, migraine, eczema, arthritis, asthma, autism, Candidiasis, celiac disease, chronic fatigue, diabetes, ear infections, fibromyalgia, hyperactivity, hypoglycemia, hypertension, leaky gut, skin rashes and, sinusitis, are each considered in some cases to be provoked or caused by intolerance to allergens present in food.

The following will illustrate conditions caused, at least in part, by food intolerance.

Irritable bowel syndrome (IBS) is a chronic bowel disorder resulting from abnormal contractions of the large intestine. These contractions lead to spasms in the colon causing abdominal pain and irregular bowel movements. The symptoms can last for several days or even months. Research into the causes of IBS have not discovered any anatomical or biochemical abnormalities in the bowel of sufferers of IBS nor are there any specific tests to determine whether a person, or animal, is susceptible to IBS. The condition is not life threatening but can cause debilitation resulting in hospitalisation and absenteeism from work. It is thought that diet has an influence on the severity of IBS and that stress can also contribute.

A further example of a condition thought to be provoked or caused by food allergy is migraine. The exact cause of migraine is uncertain but is likely to be multi-factorial. A theory currently favoured is that those suffering from the condition have inherited a more sensitive nervous system response than those who do not suffer from migraine. During a migraine attack changes in the brains activity produce inflamed blood vessels around the brain. It is known that certain triggers provoke a migrane attack. The diet of a migraine sufferer is thought to be a major trigger of migraine. These foods include alcohol, especially red wine, foods containing monosodium glutamate, and food containing tyramine (eg aged cheeses, preserved meats with nitrates and nitrites). Other factors thought to be involved include: disturbed sleeping patterns; irregular fluctuations in hormone levels (women may have attacks correlated with their menstrural cycle); stress and anxiety; and environmental factors such as weather, fluorescent lights, computer screens, strong odors and high altitude. Clearly migraine is caused by a combination of factors which are not easily controlled.

Food allergy occurs when the immune system (a combination of immune cells, antibodies and chemical mediators) reacts to an allergen present in food to remove it from an animal's system. Currently there are a number of food allergy test kits which screen an individual against a large number of potential food allergens. These test kits are expensive and require the patient to visit a hospital or doctors surgery so that a sample of blood may be taken and tested. It would be desirable if a preliminary screen could be conducted to determine if the patient would benefit from the broader screen. The preliminary screen would comprise testing a blood sample against a narrower combination of allergens which would be indicative of food intolerance. The test would be a simple positive or negative and, if positive, may encourage the patient to pay for a more expensive and extensive test to determine the specific foods which the patient should avoid.

### DETAILED DESCRIPTION OF THE INVENTION AND THE PREFERRED EMBODIMENTS

According to a first aspect of the invention there is provided a substrate to which is applied, coupled or cross-linked to at least one part of said substrate an allergen wherein said allergen is a protein derived from an extract selected from at least one of the following food groups: cereal, legume, cocoa bean, nut, fruit, vegetable, shellfish, fish, yeast, dairy product, egg and meat.

The allergen may be selected from at least 2, 4, 6, 8, 10, or from all, of said food groups.

In a preferred embodiment the allergen is a protein derived from an extract selected from each of cereal, legume, cocoa bean, nut, fruit, vegetable, shellfish, fish, yeast, dairy product, egg and meat.

Typically, the number of allergens applied, coupled or cross-linked to the substrate is between 100 and 50; preferably between 50 and 20; more preferably between 40 and 20; even more preferably about 30.

In a preferred embodiment, between 30 and 20 allergens are selected from said food groups such that at least one allergen is selected from each of said food groups.

In a preferred embodiment the cereal includes barley, corn, rice, rye and wheat. In a preferred embodiment the legume includes bean, particularly haricot bean and soybean, and pea including peanut. In a preferred embodiment the nut includes almond, brazil nut, cashew nut, walnut. In a preferred embodiment the fruit includes tomato, apple, orange, strawberry. In a preferred embodiment the vegetable includes cabbage and celery. In a preferred embodiment the dairy product includes cows milk. In a preferred embodiment the meat includes beef, chicken and pork. In a preferred embodiment the fish is white fish meat.

In a preferred embodiment the allergen includes a protein derived from an extract of each of barley, corn, rice, rye, wheat, cows milk, beef, chicken, pork, cabbage, celery, haricot bean, pea, potato, soybean, tomato, apple, orange, strawberry, almond, brazil nut, cashew nut, peanut, walnut, cocoa bean, yeast, shellfish, white fish and egg.

Typically, the allergen is provided on the substrate at a protein concentration of between about 1.0-60.0 μg/ml, preferably between 3.0-50.0 μg/ml. In an embodiment of the invention, the cereal allergen is provided at a concentration of between about 12.0-50.0 μg/ml. In an alternative embodiment, the legume allergen is provided at a concentration of between about 3.0-50 μg/ml. In an alternative embodiment, the nut allergen is provided at a concentration of between about 10.0-30 μg/ml, preferably about 20 μg/ml. In an alternative embodiment, the fruit allergen is provided at a concentration of between about 30-50 μg/ml, preferably about 50 μg/ml. In an alternative embodiment, the vegetable allergen is provided at a concentration of between about 10.0-50 μg/ml. In an alternative embodiment, the meat allergen is provided at a concentration of between about 20.0-50 μg/ml. In an alternative embodiment, the cocoa bean allergen is provided at a concentration of between 10-30 μg/ml, preferably about 20 μg/ml. In an alternative embodiment, the shellfish allergen is provided at a concentration of between 5-20 μg/ml, preferably about 10 μg/ml. In an alternative embodiment, the fish allergen is provided at a concentration of about 30-50 μg/ml, preferably about 50 μg/ml. In an alternative embodiment, the yeast allergen is provided at a concentration of between 5-20 μg/ml, preferably about 8 μg/ml. In an alternative embodiment, the dairy product allergen is provided at a concentration of between 5-20 μg/ml, preferably about 12 μg/ml. In an alternative embodiment, the egg allergen is provided at a concentration of between 5-20 μg/ml, preferably about 12 μg/ml.

In a preferred embodiment of the invention the extracts further comprise a buffer or diluent.

In a yet further preferred embodiment of the invention the allergens are associated, couple or cross-linked to a detectable label. Preferably said detectable label is biotin.

In a preferred embodiment the allergen derived from each of the extracts is arranged as an array on said substrate. Preferably, the array is a microarray or a microdot.

The substrate may be nitrocellulose, glass, modified glass or plastic.

In a yet further aspect the invention provides an assay product comprising the substrate of the present invention. The assay product may include a test tube, bead, sheet, fibre, mat or microtiter plate, and the like, made, for example, from glass, plastic or cellulosic substrates. In a preferred embodiment, the assay product is a microtiter plate.

Further provided is an assay product according to the invention for use with an array reader or array printer.

A further aspect of the invention provides a method of preparing the substrate according to the invention, comprising:
i) preparing protein extracts, to an appropriate concentration in buffer, from the food groups: cereal, legume, nut, cocoa bean, fruit, vegetable, shellfish, fish, yeast, dairy product, egg and meat;
ii) optionally preparing a dilution series of the extract solutions of (i); and
iii) loading a substrate with the extract solutions of (i) or (ii) whereby the solutions are loaded in succession or simultaneously to separate areas on the substrate;

In a preferred embodiment of the invention the substrate is loaded in (iii) as an array.

The present invention has determined the identity of the major food allergens implicated in food intolerance. Those individuals with a positive reaction to this combination of allergens would benefit from a more extensive screen against a broader range of food allergens to determine the identity of the specific food groups which should be avoided.

In a yet further aspect the invention provides a method to test whether an animal is intolerant to at least one food allergen comprising the steps of:
i.) contacting a body fluid sample from said animal with the substrate of the present invention;
ii.) measuring or detecting the binding of antibodies in said body fluid sample with the allergens on said substrate.

The body fluid may be selected from the group consisting of: blood or serum; semen; lymph fluid; cerebrospinal fluid; synovial fluid; tears; sweat; urine; saliva; or bone marrow. Preferably, said body fluid sample is blood or serum. Typically the patient directly provides the body fluid sample without the need to visit a medical practitioner.

The detection of antibody:antigen complexes is well known in the art. Typical methods involve the detection of an antibody:antigen complex using a labelled secondary antibody directed to the antibody bound to the antigen. The secondary antibody can be labelled with an enzyme (eg horse radish peroxidase; alkaline phosphatase) or a fluorescent label (eg fluoresceine, rhodamine) or with gold particles. Alternatively, the antibodies present in the serum can be directly labelled followed by incubation with the allergen. This type of assay is referred to as an Enzyme Linked ImmunoSorbant Assay (ELISA) or Enzyme Linked Immunoassy (ELA).

A preferred method according to the invention is the use of the so-called sandwich immunoassay. This involves mixing a body fluid sample with a biotin labelled food allergen and with gold-labelled avidin. The avidin has a high affinity for the biotinylated allergen. Bivalent antibodies which bind the biotin:avidin allergen complex present in the body fluid sample bind to the allergen present on the test substrate. The gold label serves as a visualisation agent. The sandwich method provides for a sensitive assay for the presence of allergen specific antibodies since only when the antibody forms a bridge between the biotin:allergen and the avidin:gold is a positive result obtained.

In a further preferred method of the invention said method detects an immunoglobulin. Preferably said immunoglobulin is selected from the following Ig isotypes: IgA, IgM, IgD, IgE and IgG. In a yet further preferred method of the invention said immunoglobulin is IgG. Preferably said IgG is selected from the group consisting of: IgG1, IgG2, IgG3 or IgG4.

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

In a further preferred method according to the invention said body fluid sample is combined with a biotinylated food allergen to which is further added avidin which is provided with a detectable label. Preferably said label is gold.

According to a further aspect of the invention there is provided a kit comprising a substrate according to the invention; detection means for the detection or measurement of allergen: antibody complexes; buffers and cofactors.

According to a further aspect of the invention there is provided an immunoassay as herein described with reference to the description and drawings.

An embodiment of the invention will now be described by example only:

### EXAMPLES

### Example 1

Selection of Food Allergens for Inclusion in the Test Panel

The food allergens for inclusion in the panel were selected from an examination of the frequency of IgG positivity in preliminary experiments with a larger panel of food allergens. The following 29 food allergens were selected:

| | | |
|---|---|---|
| Barley | Celery | Brazil nut |
| Corn | Haricot Bean | Cashew nut |
| Rice | Pea | Peanut |
| Rye | Potato | Walnut |
| Wheat | Soybean | Cocoa bean |
| Cows Milk | Tomato | (Brewer's) Yeast |
| Beef | Apple | Shellfish mix |
| Chicken | Orange | White fish mix |
| Pork | Strawberry | Whole egg |
| Cabbage | Almond | |

### Example 2

Preparation of the 29 Food Panel ELISA Test Plates

Materials:

Sodium carbonate/bicarbonate buffer pH 9.6, 0.05M (Sigma) (Coating buffer). Food allergen extracts (Antigen Laboratories Inc and Greer Laboratories Inc, USA). Phosphate buffered saline (PBS)+ProClin 300™ preservative, 0.05% v/v (Supelco, USA). ELISA plates, 1×8 well single break strip, high bind, flat bottomed (Greiner, Germany). MPCS3 microplate conveyor system (Oyster Bay Inc, USA). PBS+fish gelatin,0.2% w/v+sucrose, 0.05% w/v+Proclin 300, 0.05% v/v (Blocking Buffer). PBS+Tween 20™ detergent, 0.05% (Wash Buffer). Adjustable pipettes and pipette tips, 200 ul, 1000 ul and 5000 ul.

Method:

Allergen extracts were diluted to the appropriate concentration in Coating Buffer to 40 ml, sufficient for the preparation of 400 ELISA test strips (see table below). All subsequent additions of solutions to and aspiration of solutions from the ELISA plate were performed on a MPCS microplate conveyor system.

100 ul of each diluted allergen extracts for the test and control wells were added to each well in 4×8 well single break strips (A, B, C and D) according to the co-ordinates shown in the table below. The ELISA plates loaded with food allergen extract solutions in coating buffer were incubated overnight at 2-8° C. Following incubation, the charged ELISA plates were aspirated and washed for three cycles with Washing Buffer at 450 ul per well and vacuumed dry. 300 ul of Blocking Buffer was added to all well and the plates incubated for 60 minutes at room temperature (21° C.). Following incubation, the charged ELISA plates were aspirated to dryness and incubated at 37° C. for a further 120 minutes to ensure plates were completely dry. For final assembly, 3 strips of each of the A, B, C and D strips were assembled into a single test containing 12 strips, with 8 wells per strip (as three repeats of four strips). The allergen coated plates were stored within sealed foil pouches with desiccant at 2 to 8° C. until required.

| | | | | | | Vol |
|---|---|---|---|---|---|---|
| | | | | | Required | Allergen |
| | | | | Starting | Coating | soln |
| | | Food | | conc | Level | to add |
| Strip | Row | Extract ID | Source | (mg/ml) | (ug/ml) | (ul) |
| A | A | Cows Milk | A | 4.86 | 12.5 | 103 |
| | B | No coat - | | N/A | | |
| | | blocker only | | | | |
| | C | Barley | A | 2.03 | 12.5 | 246 |
| | D | Corn | A | 3.25 | 12.5 | 154 |
| | E | Rice | A | 1.77 | 20.0 | 452 |
| | F | Rye | G | 27.44 | 50.0 | 73 |
| | G | Wheat | A | 3.59 | 12.5 | 139 |
| | H | Cows Milk | A | 4.86 | 12.5 | 103 |
| B | A | Beef | G | 21.32 | 50.0 | 94 |
| | B | Chicken | G | 21.36 | 30.0 | 56 |
| | C | Pork | G | 18.56 | 20.0 | 43 |
| | D | Cabbage | G | 19.28 | 50.0 | 104 |
| | E | Celery | G | 14.84 | 50.0 | 135 |
| | F | Haricot Bean | G | 20.28 | 3.0 | 6 |
| | G | Pea | G | 24.24 | 50.0 | 83 |
| | H | Potato | A | 2.41 | 13 | 207 |
| C | A | Soybean | G | 17.52 | 50.0 | 114 |
| | B | Tomato | G | 16.24 | 50.0 | 123 |
| | C | Apple | G | 14.2 | 50.0 | 141 |
| | D | Orange | G | 16.84 | 50.0 | 119 |
| | E | Strawberry | G | 11.60 | 50.0 | 172 |
| | F | Almond | G | 20.68 | 20.0 | 39 |
| | G | Brazil nut | G | 28.68 | 20.0 | 28 |
| | H | Cashew nut | G | 19.28 | 20.0 | 41 |
| D | A | Peanut | G | 31.56 | 25.0 | 32 |
| | B | Walnut | G | 27.76 | 20.0 | 29 |
| | C | Cocoa bean | G | 47.72 | 20.0 | 17 |
| | D | Brewers Yeast | A | 7.10 | 8.0 | 45 |
| | E | Shellfish Mix | G | 37.01 | 10.0 | 11 |
| | F | Fish mix | G | 34.09 | 50.0 | 59 |
| | G | Whole egg | G | 34.72 | 12.5 | 14 |
| | H | No coat - | | N/A | | |
| | | blocker only | | | | |

A = Antigen Laboratories
G = Greer Laboratories

Screening Patient Specimens
Assay Principle:

The Foodscan food intolerance ELISA test is designed to detect and measure qualitatively IgG antibodies to food allergen extracts in dilute human serum, plasma and whole blood. Food allergen extracts from a panel of 29 foodstuffs were coated onto the surface of four strips, with eight allergens per strip. Test specimens were pre-diluted to 1/50, 1/150 and 1/450, with each dilution applied to a group of four allergen panel strips in singlicate. Each test was calibrated using 0 arbitrary unit (AU) and 25 AU calibrators prepared from a pool of high titre cow's milk allergen-specific IgG positive serum. A positive control (45 AU) was applied to each test. The ELISA plate plus specimen is incubated for a brief period during which IgG antibodies specific to a particular food allergen will bind to that allergen in the specific well. After incubation, the plate is washed to remove irrelevant antibodies and other serum components. The specific anti-food antibodies bound to the food allergens are detected by an anti-human IgG antibody horseradish peroxidase conjugate. The action of the bound peroxidase activity on a clear solution of an enzyme substrate generates a blue coloured product, converted to an intense yellow colour on addition of acid solution to stop the reaction. The colour developed in each well is proportional to the amount of food allergen specific antibody in the original test sample. Test results were obtained from the 1/150 dilution of the specimen. Where a high specimen background was observed the test results were obtained from the 1/450 (higher) dilution.

### Example 3

Preparation of the 29 Food Panel ELISA Test Plates

Materials:

29 food allergen coated ELISA plate. PBST, 0.05%+PVP 10k, 0.1% +Proclin 300™ (Sample Diluent). 0 U/ml and 25 U/ml calibrator and positive controls, prediluted to working strength. PBST, 0.05% (Wash Buffer). Goat anti-human IgG (Fc Specific)—horse radish peroxidase enzyme conjugate (Sigma), diluted 1:4000 in Stabizyme/UHQ Water 1:1 (Surmodics Inc, USA) (Conjugate Solution). 3,3′,5,5′ tetramethylbenzidene peroxidase substrate TMB one-component substrate (K+P Laboratories Inc, USA). H₂SO₄, 0.5M (Riedel de Haan, Germany) (Stop Solution). Titramax 100 ELISA plate shaker (Heidolph, Germany). MRX Microtiter Plate Reader with Absorbance Reader at 450 nm wavelength and Revalation™ data analysis software (Dynex, USA). Ultrawash ELISA plate washer (Dynex, USA).

Method:

All Materials except Wash buffer and Stop solution were pre-prepared and stored at 2-8° C. prior to use. All materials were allowed to warm to room temperature before use. 100 ul calibrators and control were into the specified wells in strip A as follows:
A1 (first set)—25 U/ml Calibrator
A1 (second set)—25 U/ml Standard
A1 (third set)—Positive Control

100 ul of patient sera pre-diluted as described above was added to the plate; 1/50 dilution to the first set of four strips A-D, the 1/150 dilution to the second series of strips A-D and 1/450 to the third series of strips A.

The test plate charged with test specimen, calibrator and control was incubated for 30 minutes at room temperature with shaking on the ELISA plate shaker at setting 10 (max). The test plate was washed 6 times with Wash Buffer and aspirated to dryness.

100 ul of the prediluted Conjugate Solution was added to all test plate wells. The test plate was incubated for a further 30 minutes at room temperature without shaking.

The test plate was washed 6 times with Wash Buffer and aspirated to dryness. 100 ul of TMB Substrate Solution was applied to all the wells. The plate was incubated for a further 10 minutes at room temperature without shaking. The reaction was stopped with the addition of 50 ul of Stop Solution to all the wells. The absorbance due to the colour developed in the test, calibrator and control wells was read at 450 nm within 10 minutes using the Plate Reader.

Result Calculation:

The results of each test were regarded as qualitative. The threshold for a positive (reactive) result was selected as three times the sample background sample against no food allergen coated test well, equivalent to 3.0 arbitrary units. Test results were scored as positive or negative only relative to the cutoff.

What is claimed is:

1. A substrate to which is applied, coupled or cross-linked to at least one part of said substrate an allergen wherein said allergen is a protein derived from an extract which is a member selected from at least one of the following food groups: cereal, legume, cocoa bean, nut, fruit, vegetable, shellfish, fish, yeast, dairy product, egg and meat.

2. The substrate according to claim 1 wherein the allergen is a member selected from at least 2, 4, 6, 8, 10, and from all, of said food groups.

3. The substrate according to claim 1 wherein the allergen is a protein derived from an extract which is a member selected from each of cereal, legume, cocoa bean, nut, fruit, vegetable, shellfish, fish, yeast, dairy product, egg and meat.

4. The substrate according to claim 1 wherein the number of allergens applied, coupled or cross-linked to the substrate is an integer from 50 to 100.

5. The substrate according to claim 4 wherein said number of allergens is an integer from 20 to 50.

6. The substrate according to claim 5 wherein said number of allergens is an integer from 20 to 30.

7. The substrate according to claim 1 wherein the allergens are selected from said food groups such that at least one allergen is selected from each of said food groups.

8. The substrate according to claim 1 wherein the cereal is a member selected from barley, corn, rice, rye and wheat.

9. The substrate according to claim 1 wherein the legume is a member selected from bean, soybean and pea.

10. The substrate according to claim 1 wherein the nut is a member selected from almond, brazil nut, cashew nut and walnut.

11. The substrate according to claim 1 wherein the fruit is a member selected from tomato, apple, orange and strawberry.

12. The substrate according to claim 1 wherein the vegetable is a member selected from cabbage and celery.

13. The substrate according to claim 1 wherein the dairy product is cows milk.

14. The substrate according to claim 1 wherein the meat is a member selected from beef, chicken and pork.

15. The substrate according to claim 1 wherein the fish is white fish meat.

16. The substrate according to claim 1 wherein the allergen includes a protein derived from an extract of each of barley, corn, rice, rye, wheat, cows milk, beef, chicken, pork, cabbage, celery, haricot bean, pea, potato, soybean, tomato, apple, orange, strawberry, almond, brazil nut, cashew nut, peanut, walnut, cocoa bean, yeast, shellfish, white fish and egg.

17. The substrate according to claim 1 wherein the allergen is provided on the substrate at a protein concentration of between about 1.0-60.0 μg/ml.

18. The substrate according to claim 17 wherein the allergen is provided on the substrate at a protein concentration of between 3.0-50.0 μg/ml.

19. The substrate according to claim 17 wherein the cereal allergen is provided on the substrate at a protein concentration of between about 12.0-50.0 μg/ml.

20. The substrate according to claim 17 wherein the legume allergen is provided on the substrate at a protein concentration of between about 3.0-50 μg/ml.

21. The substrate according to claim 17 wherein the nut allergen is provided on the substrate at a protein concentration of between about 10.0-30 μg/ml.

22. The substrate according to claim 17 wherein the fruit allergen is provided on the substrate at a protein concentration of between about 30-50 μg/ml.

23. The substrate according to claim 17 wherein the vegetable allergen is provided on the substrate at a protein concentration of between about 10.0-50 μg/ml.:

24. The substrate according to claim 17 wherein the meat allergen is provided on the substrate at a protein concentration of between about 20.0-50 μg/ml.

25. The substrate according to claim 17 wherein the cocoa bean allergen is provided on the substrate at a protein concentration of between 10-30 μg/ml.

26. The substrate according to claim 17 wherein the shellfish allergen is provided on the substrate at a protein concentration of between 5-20 μg/ml.

27. The substrate according to claim 17 wherein the fish allergen is provided on the substrate at a protein concentration of about 30-50 μg/ml.

28. The substrate according to claim 17 wherein the yeast allergen is provided on the substrate at a protein concentration of between 5-20 μg/ml.

29. The substrate according to claim 17 wherein the dairy product allergen is provided on the substrate at a protein concentration of between 5-20 μg/ml.

30. The substrate according to claim 17 wherein the egg allergen is provided on the substrate at a protein concentration of between 5-20 μg/ml.

31. The substrate according to claim 1 wherein the extract(s) further comprise a buffer or diluent.

32. The substrate according to claim 1 wherein the allergens are associated, coupled or cross-linked to a detectable label.

33. The substrate according to claim 32 wherein the detectable label is biotin.

34. The substrate according to claim 1 wherein the allergen derived from the extract(s) is arranged as an array on said substrate.

35. The substrate according to claim 34 wherein the array is a microarray or a microdot.

36. The substrate according to claim 1 wherein the substrate is a member selected from nitrocellulose, glass, modified glass and plastic.

37. An assay product comprising a substrate according to claim 1.

38. The assay product according to claim 37 wherein said assay product further comprises a member selected from a test tube, bead, sheet, fibre, mat and microtiter plate.

39. The assay product according to claim 38 wherein said assay product is manufactured from a material which a member selected from glass, plastic and cellulosic substrates.

40. The assay product according to claim 38 wherein said assay product is a microtiter plate.

41. The assay product according to claim 37 for use with a member selected from an array reader and an array printer.

42. A method of preparing a substrate according to claim 1 the method comprising:
i) preparing protein extracts from a food group which is a member selected from cereal, legume, nut, cocoa bean, fruit, vegetable, shellfish, fish, yeast, dairy product, egg and meat;
ii) optionally preparing a dilution series of the extract solutions of (i); and
iii) loading a substrate with the extract solutions of (i) or (ii) whereby the solutions are loaded in succession or simultaneously to separate areas on the substrate.

43. The method according to claim 42 wherein the substrate is loaded in (iii) as an array.

44. A method to test whether an animal is intolerant to at least one food allergen comprising the steps of:
i) contacting a body fluid sample from said animal with a substrate as claimed in claim 1; and
ii) measuring or detecting the binding of antibodies in said body fluid sample with the allergens on said substrate.

45. The method according to claim 44 wherein the body fluid is a member selected from blood, serum, semen, lymph fluid, cerebrospinal fluid, synovial fluid, tears, sweat, urine, saliva and bone marrow.

46. The method according to claim 44 wherein the method is an immunoassay.

47. The method according to claim 46 wherein the immunoassay detects an immunoglobulin.

48. The method according to claim 47 wherein the immunoglobulin is IgG.

49. The method according to claim 48 wherein the IgG is a member selected from IgG1, IgG2, IgG3 and IgG4.

50. The method according to claim 44 wherein the body fluid sample is combined with a biotinylated food allergen to which is added avidin provided with a detectable label.

51. An immunoassay comprising:
a substrate and
an allergen
wherein
said allergen is applied, coupled or cross-linked to at least one part of said substrate and
said allergen is a protein derived from an extract which is a member selected from at least one of the following food groups: cereal, legume, cocoa bean, nut, fruit, vegetable, shellfish, fish, yeast, dairy product, egg and meat.
